(19) United States
(12) Patent Application Publication
(10) Pub. No.: US 2003/0017158 A1
(43) Pub. Date: Jan. 23, 2003

(54) Method for detecting and killing epithelial cancer cells

(76) Inventors: Samuel D. Bernal, Woodland Hills, CA (US); Douglas D. Burkett, Gilbert, AZ (US); Ralph E. Green, Scottsdale, AZ (US); Seth D. Rose, Tempe, AZ (US)

Correspondence Address: William H Drummond, Drummond & Duckworth, Suite 500, 4590 MacArthur Boulevard, Newport Beach, CA, 92660, US

(21) Appl. No.: 10/019,494

(22) PCT Filed: Feb. 27, 2001
(86) PCT No.: PCT/US01/06318

(51) Int. Cl. A61K 39/395; G01N 33/574

(52) U.S. Cl. 424/155.1; 435/7.23

(57) ABSTRACT

A diagnostic method for detecting cancerous epithelial cells by selectively marking the mitochondria of the cancer cells, by delivering to the epithelium a cationic supravital mitochondrial marking agent. Selective killing of cancerous epithelial cells in the presence of normal cells is effected by delivering a cationic supravital mitochondrial marking agent to epithelial cancer cells. The killing agent can also comprise the reaction product of the marking agent and a cancer chemotherapeutic drug or in admixture

### RELATED APPLICATION

This application is a continuation-in-part of copending International Application, PCT/US00/05387, tiled Feb. 28, 2000, entitled “Method of Detecting and Killing Epitheleal Cancer Cells.

### FIELD OF THE INVENTION

This invention relates to methods for detecting epithelial cancer.

In another respect the invention pertains to methods for selectively killing epithelial cancer cells.

In a further aspect, the invention concerns methods for detecting epithelial cancer cells in the presence of normal cells and/or for selectively killing such cells, in which the mitochondria of cancer cells retain a mitochondrial marking agent for a time sufficient to permit identification and/or killing such cells.

Definitions

As used herein, the following terms have the indicated meanings:

“Cancer” or “cancerous” cells are used in the broad sense, to include invasive cancer cells, cancer-in-situ cells and severely dysplastic cells.

“Mitochondrial marking agent” means a compound that is selectively taken up by the mitochondria of living cancer cells and is selectively retained in cancer cells for a time sufficient to permit identification and/or killing or incapacitation thereof.

“Killing” of cells means either causing cell deaths apoptosis or cell changes that render a cell incapable of reproduction and metastasizing.

“Adduct” means the reaction product, either covalent or noncovalent, of a mitochondrial marking agent and a cancer chemotherapeutic agent.

“Adjuvant” means a mitochondrial marking agent that, in combination with another chemotherapeutic agent, causes improved killing of cancer cells, either synergistically or by additive effects with the other agent.

### BACKGROUND OF THE INVENTION

In-vivo diagnostic procedures for detecting malignant and premalignant epithelial lesions or carcinomas, employing dye compositions that selectively “color” tissues that are abnormal due to dysplasia, hyperplasia, tumorigenesis and other active surface lesions, are known in the art. These diagnostic methods employ a dye that imparts color to a cancerous substrate, which is then detectable under light at visible wavelengths or a fluorescent dye that imparts color to the substrate, which is then detectable when illuminated by light at wavelengths outside the visible spectrum.

For example, procedures employing fluorescein and fluorescein derivatives are disclosed in Chenz, Chinese Journal of Stomatology (27:44-47 (1992)) and Filurin (Stomatologiia (Russian) 72:44-47 (1993)). These procedures involve application of the dye, followed by examination under ultraviolet light to detect the cancerous/precancerous tissue, which is selectively fluorescent. Another prior art procedure involves rinsing the epithelium with toluidine blue, followed by normal visual examination to detect any selectively stained tissue. Such procedures are disclosed, for example in the patents to Burkett (U.S. Pat. No. 6,086,852), Tucci (U.S. Pat. No. 5,372,801) and Mashberg (U.S. Pat. No. 4,321,251). Use of other thiazine dyes and oxazine dyes in an analogous manner is disclosed in U.S. Pat. No. 5,882,627 to Pomerantz.

Heretofore, it was theorized that such dyes selectively “marked” cancerous tissue because the dye was retained in the relatively larger interstitial spaces between the cells of cancerous tissue and would not efficiently penetrate the tighter intracellular junctions of normal tissue or be selectively retained in such relatively smaller spaces.

Contrary to the belief that toluidine blue selectively marks cancerous epithelial tissue because it is selectively retained in the relatively larger interstitial spaces between cancer cells, the mechanism of such selective staining of epithelial tissue by cationic dyes, e.g., dyes such as rhodamine, fluoresceins, oxazine and thiazine dyes (including toluidine blue) and other cationic supravital marking agents, is the selective uptake and selective retention of the agent in the mitochondria of cancer cells. This selective mitochondrial uptake and retention is apparently due to the higher electrical potential (negative charge on the inside of the membrane) of cancerous cells' mitochondria as compared to mitochondria of normal cells. See, e.g., Chen et al., Cancer Cells 1/The Transformed Phenotype, 75-85 (Cold Spring Harbor Laboratory, 1984); Lampidis, et al., Cancer Research 43, 716-720 (1983). In fact, the selective marking of cancer cells by, and retention in the mitochondria of cancer cells of, supravital cationic dyes and other supravital cationic marking agents, are related to one of the very characteristics of cancer cells that appears to be responsible for their rapid cloning growth and metastasizing ability, namely, that the higher electrical potential of the mitochondria of cancer cells is the source of cellular energy and is the driving force for ATP (adenosine triphosphate)production by the cells.

### SUMMARY OF THE INVENTION

We have now discovered a method for in-vivo detection of cancerous epithelial cells by selective marking of the mitochondria thereof.

In another respect, we have discovered a therapeutic method for selectively killing cancer cells in the presence of normal cells.

Our detection methods comprise the steps of delivering a cationic supravital mitochondrial marking agent to tissue in the locus of a suspect cancerous site on the epithelium (which contains both normal and cancerous cells), thus causing said agent to be taken up and selectively retained in the mitochondria of the cancer cells. The cancerous cells are then detectable by any suitable method, for example, instrumental or visual examination under visible light or under light of selected invisible wavelengths.

In a further embodiment, after the marking agent is taken up by the mitochondria, a rinse reagent is applied to the locus of the suspect cancerous site, thus enhancing the rate of release of the agent from the mitochondria of the normal cells and further increasing the selectivity of the diagnostic methods.

According to another important embodiment of the invention, we provide a method for selectively killing cancerous epithelial cells comprising the step of contacting cancerous cells in the locus of a suspect cancerous site with a cationic supravital mitochondrial marking agent, to cause cell death or to render the cancer cells substantially incapable of multiplication. The marking agent can be delivered to the cancer cells in a single discrete dose, or continuously, or in repeated discrete doses, with or without employing a rinse reagent after each dose.

In a further embodiment of the invention, we provide a method of improving the selectivity and cancer cell killing ability of cancer chemotherapeutic agents comprising the steps of either (1) forming a reaction product of a cationic supravital agent and a chemotherapeutic agent and delivering the reaction product to cancerous epithelial cells or (2) combining the cationic supravital agent with a cancer chemotherapeutic agent, to improve the selectivity or killing ability of the chemotherapeutic agent, either by additive or synergistic effects, or both.

These, other and further embodiments of the invention will be apparent to those skilled in the art and a better understanding of the invention will be obtained from the following examples which are provided to illustrate the invention and not as indications of the scope thereof, which is defined only by the appended claims.

In the practice of the invention and in the following working examples, cationic supravital mitochondrial marking agents, include dyes, including toluidine blue O, alcian blue, malachite green, phenosafranin, acriflavine, pyronine Y, toluylene blue and brilliant green; and

“non-dye” compounds, including peonidin, oxythiamine, tiemonium iodide, elliptinium acetate and furazolium chloride.

According to the presently preferred embodiment of the invention, the preferred mitochondrial marking agents are dyes of the oxazine and thiazine class. The thiazine dyes are especially preferred, particularly toluidine blue O, Azure A, Azure B and ring-substitution and N-substitution analogs thereof.

In order to be selectively absorbed and retained in cancer cell mitochondria, the marking agent or reaction product of marking agent+chemotherapeutic agent, must have a molecular weight of below about 5,000. Further, because of marked differences in the selective marking and therapeutic activity of various closely related analogs, it appears that the molecular structure of the marking agent significantly affects its ability to selectively mark and/or kill living cancer cells in the presence of normal living cells. These differences in cell marking and killing ability are related to structural features, e.g., location and type of ring-substituents and N-substituents, of the marking agent molecules that implicate one or more or all of the following mechanisms of action:

1. The structure of the marking agent molecule, e.g., position and nature of ring and N-substituents on the cationic molecule, affects the availability of the positive charge and hinders the ability of the marking agent or “stacked” groups of them to be attracted by the negative charges on the mitochondrial membranes or within the mitochondria.

2. The structure of the marking agent molecule permits it to intercalate into or “stack” along the exterior of mitochondrial DNA of cancer cells.

3. The structure of the marking agent molecule permits it or stacked groups of them to bind to specific active sites, e.g., four specific proteins, in the mitochondria, and/or precipitate with cardiolipins at the inner surface of the mitochondrial membrane.

4. The structure of the marking agent affects its reduction potential and its tendency to change to the uncharged “leuco” form.

5. The structure of the marking agent affects its acidity (pKₐ), and, in turn, the ability of the cationic marking agent to deprotonate at physiological pH. Thus, the cationic form of the dye can be attracted to the outer surface of the mitochondrial membrane, whereupon the dye cation can lose a proton and concomitantly lose its positive charge, thereby liberating the neutral form of the dye, which may more readily penetrate the nonpolar matrix of the membrane and gain access to the interior of the mitochondrion.

The intermolecular interactions of mechanisms 1 dye-membrane), 2 (dye-base pair or dye-dye), and 3 (dye-protein or dye-lipid) depend on the hydrophobicity-lipophilicity of the dye, which can be assessed by various means, one of which is the partition coefficient between aqueous solution and a low-polarity organic solvent, such as 1-octanol (i.e., log P values). Mechanisms 4 and 5 depend on hydrophobicity-lipophilicity, due to the effect of differential solvation of reactant and product on reduction potential (oxidized vs. reduced forms) and pKₐ (neutral vs. charged forms). For example, hydrophobicity hampers the solvation of protonated tertiary aliphatic amines (R₃NH⁺), thereby decreasing their acidity relative to secondary amines (R₂NH₂⁺)

According to the presently preferred embodiment of the invention, one employs a cationic supravital marking agent having a log P of from about −1.0 to about 5.

The following examples are presented to enable those skilled in the art to understand and practice the invention and to identify the presently preferred embodiment. These examples are for illustrative purposes only are not intended to limit the scope of the invention, which is defined only by the appended claims.

### EXAMPLE 1

### Uptake and Retention of Mitochondrial Marking Agents in Living Carcinoma Cells

Different concentrations of the various cationic marking agents, at 100, 50, 10 and 1 μg/ml are prepared in RPMI medium complete with 20% fetal calf serum, 1 mM glutamine, hydrocortisone, insulin, transferrin, estradiol, selenium and growth hormone.

The carcinoma cells are incubated at 37EC in tissue culture incubators with 5% CO₂ and 95% relative humidity, for 5 minutes with each agent and concentration there and then rinsed twice using 2 minute incubations with 1% acetic acid. After incubation and rinsing, the cells are harvested, at 30 min., 1 hour, 2 hours, 4 hours and 8 hours. The cells are then extracted with 2-butanol and analyzed by spectrophotometry for quantitation of the marking agent.

The results show that there is a concentration dependence in the rate of accumulation of marking agent in the mitochondria of both carcinoma and normal cells and in the selectivity of release of the marking agent from cancer cells, but this concentration dependence starts to become less pronounced. The saturation concentration for toluidine blue O occurs at concentrations of 10 μg/ml and above. The saturation concentrations for the other marking agents are similarly determined. The remaining experiments are conducted with a concentration of 10 μg/ml for toluidine blue O and at the saturation concentrations for the other marking agents so-determined, unless stated otherwise.

### EXAMPLE 2

### Mitochondrial Localization of the Agents in Living Cells

After incubation and rinsing of various cell lines, using the different cationic marking agents, the mitochondrial localization of the agents is analyzed using confocal high resolution microscopy and phase contrast microscopy.

Living cells, are cultivated in complete growth medium with 20% fetal calf serum and growth factors, and maintained at 37EC. These cells accumulate and retain the marking agents in the mitochondria. When these cells are then maintained in an agent-free medium, carcinoma cells retain the agent for longer than about 1 hour, but normal epithelial cells release the agent within about 15 minutes.

In contrast to living cells, dead cells or cells treated with agents that dissipate the mitochondrial membrane potential lose mitochondrial staining and accumulate the agents in the nucleus.

### EXAMPLE 3

### Release of the Agents from Mitochondria with Dissipation of the Mitochondrial Membrane Potential

Known agents that alter the mitochondrial electrical potential are used to pretreat epithelial cancer cells, followed by treatment with the cationic supravital mitochondrial marking agents. These pretreatment agents include azide and cyanide preparations and dinitrophenol.

Epithelial cancer cells are also pre-stained with the various dyes and then are post-treated with these known agents. The release of the dyes from the cells or the transfer of the dyes to other subcellular compartments, including the nucleus is analyzed.

The cells pretreated with these agents do not accumulate dyes in the mitochondria and the mitochondria of the pre-stained cells release the dye upon post-treatment with these agents.

### EXAMPLE 4

### Tissue Explants of Squamous Carcinomas

Fresh explants of resected epithelial carcinomas are analyzed for marking agent uptake and retention. After resection, the carcinomas are microdissected from surrounding tissue, cut into 3 mm sections and maintained as explant tissue cultures at 37° C. These explants are then incubated with the various agents and then extracted for quantitation of the agent.

Oral carcinoma (SqCHN) have rapid uptake and prolonged retention of these agents. The agents start to be released from the cells after about one hour of cultivation in agent-free medium. However, the agents are released faster when the cells are incubated in medium that does not contain growth factors, fetal calf serum and other medium additives. The agents are also released faster when the cells are grown in adverse conditions such as lower temperatures.

### EXAMPLE 5

### Tissue Explants of Normal Epithelial Cells

Cells obtained surgically from normal areas of the oral epithelium are cultivated as normal epithelial cultures. These cultures are then incubated with the marking agents for analysis of the agent uptake and retention.

Unlike the carcinoma cells, normal epithelial cells quickly release the agents from their mitochondria and from the cell much more quickly. By 10-15 minutes, most of the agent is released from the mitochondria.

### EXAMPLE 6

### Marking Agent-Chemotherapeutic Agent Adducts

In place of the agents of Examples 1-5, the following adducts of cationic mitochondrial marking agents and various known chemotherapeutic agents are employed, with substantially similar results, except that the cancer cell kill rate and selectivity of the chemotherapeutic agent are substantially improved.

| Marking Agent | Chemotherapeutic Agent |
|---|---|
| toluidine blue O | methotrexate |
| rhodamine 123 | nitrogen mustard |

### EXAMPLE 7

### Adjuvant Compositions

The following combinations of known cancer chemotheraputants with mitochondrial marking agents exhibit synergistic or at least additive cancer cell killing effects:

| Chemotherapeutant | Cationic Marking Agent |
|---|---|
| taxol | toluidine blue |
| taxotere | azure A |
| vincristin | alcian blue |

### Selective Therapeutic Effects

In the following examples, a toluidine blue drug substance is prepared in accordance with the manufacturing procedures disclose in the U.S. Pat. No. 6,086,852, issued to Burkett on Jul. 11, 2000. Components of the drug substance are then fractionated and separated by semi-preparative HPLC, yielding the analogs identified in the '852 patent as represented by Peaks 5, 6, 7 and 8. The compounds represented by peaks 7 and 8 are toluidine blue regioisomers, having the ring methyl group in the −2 position (peak 8) and the −4 position (peak 7). The compound represented by peak 5 is the N-demethylated derivative of peak 7 and the compound represented by peak 6 is the N-demethylated derivative of peak 8.

### EXAMPLE 8

The compounds represented by peaks 5, 6, 7 and 8, obtained during the fractionation of the toluidine blue O, are analyzed for their selective toxicity towards living oral carcinoma cells (SqCHN) compared to living normal oral epithelial cells. Separate cultures of squamous carcinoma cells and normal epithelial cells are incubated with the different dye fractions and then washed with dye-free medium. The cells are then re-incubated in growth medium and observed over a period of 8 days to determine the extent of cell killing. The compound of peak 6 results in 95% cell death of carcinoma cells, compared to only about 20% killing of normal cells. The compound of peak 8 shows 89% cell death of carcinoma cells whereas it only causes about 20% killing of normal cells. Thus, the selective retention of the compounds of peaks 6 and 8 is selectively toxic towards carcinoma cells.

The selective introduction into the mitochondria of cationic dyes leads to disruption of the mitochondrial electrical potential which is the source of cellular energy and the driving force of ATP (adenosine triphosphate) production of the cells. The ability of carcinoma cells to divide rapidly and to metastasize is dependent upon the availability of this higher energy source.

However, effects on electric charge do not appear to be the only mechanism involved, because the compounds represented by peak 5 and peak 7 also are cationic dyes and yet they do not exhibit the same selective toxicity towards carcinomas that Peak 6 and Peak 8 demonstrate. Thus, the compounds of peaks 6 and 8 appear to have other molecular properties that lead to their selective toxicity towards carcinoma cells.

### EXAMPLE 9

The therapeutic characteristics of the compounds of peaks 6 and 8 are determined by further in-vitro tests, conducted in the manner of Example 8, using other isolates of carcinoma cells and normal epithelial cells. The testing profile includes other squamous carcinomas of the head and neck, esophagus, lung, cervix and skin, as well as other types of cancers, including adenocarcinomas, lymphomas and sarcomas. In-vivo “delay of tumor growth” and “tumor regression assay” tests using tumor-bearing animals, including head and neck and lung carcinomas implanted in Balb-C mice, are made to analyze the in-vivo therapeutic benefit of these compounds. These further in vitro and in-vivo tests confirm the selective toxicity of the compounds of peaks 6 and 8 to the wider variety of cancer cell types.

Having described the invention in such manner as to enable those skilled in the art to understand and practice it and, having identified the presently preferred embodiments thereof,

We claim:

1. A diagnostic method for in vivo detection of cancerous epithelial cells by selective marking of the mitochondria thereof, comprising the step of delivering to the epithelium a cationic supravital mitochondrial marking agent.

2. A method for selective killing of epithelial cancer cells comprising the step of delivering to epithelial cancer cells a cationic supravital mitochondrial marking agent.

3. The method of claim 2 in which the agent is the reaction product of a cationic supravital mitochondrial marking agent and a cancer chemotherapeutic drug.

4. The method of claim 2 in which the agent is delivered to epithelial cancer cells in combination with another cancer chemotherapeutic drug that selectively kills cancer cells by a different mechanism than the mechanism by which the agent kills cancer cells.

5. The methods of claims 1 or 2, in which the cationic supravital mitochondrial marking agent is selected to provide a molecular structure that does not hinder attraction of the positive charge of the marking agent molecule by the negative charges on the mitochondrial membranes.

6 The methods of claims 1 or 2, in which the cationic supravital mitochondrial marking agent is selected to provide a molecular structure that permits the marking agent to bind to a specific site in the mitochondria.

7. The methods of claims 1 or 2, in which the cationic supravital mitochondrial marking agent is selected to provide a structure that will intercalate into or stack along the mitochondrial DNA.

8. The methods of claims 1 or 2, in which the cationic supravital mitochondrial marking agent is selected to provide a molecular structure that affects its reduction potential to permit it to change to the uncharged leuco form prior to, during, or after entry into the mitochondria.

9. The methods of claims 1 or 2, in which the cationic supravital mitochondrial marking agent is selected to provide a molecular structure that will deprotonate at physiological pH.

10. The methods of claims 1 or 2, in which the cationic supravital mitochondrial marking agent has a log P of 0-5.
